(19) United States
(12) Patent Application Publication
(10) Pub. No.: US 2004/0101488 A1
(43) Pub. Date: May 27, 2004

(54) Method for the prevention of septic shock lethality using curcumin

(76) Inventors: Balaram Ghosh, Delhi (IN); Babita Gupta, Delhi (IN)

Correspondence Address: LADAS & PARRY, 26 WEST 61ST STREET, NEW YORK, NY, 10023, US

(21) Appl. No.: 09/535,390

(22) Filed: Mar. 24, 2000

(51) Int. Cl. A61K 7/16

(52) U.S. Cl. 424/49

(57) ABSTRACT

The present invention provides a method of preventing the lethality and reducing the severity of symptoms associated with septic shock such as lethargy, diarrhea and watery eyes induced by challenging with lipolysaccharide, in an animal in need of such a treatment by administering to the said animal a pharmacologically effective dose of curcumin orally. Also provided is a method of inhibiting the transmigration and infiltration of neutrophils from blood vessels to the underlying tissue and hence preventing the damage to the tissue of an animal in need of such a treatment by administering to the said animal a pharmacologically effective dose of curcumin orally. Curcumin being a natural compound may have little side effects. Thus, curcumin can be used to prevent the pathological conditions arising due to excessive infiltration of neutrophils into the tissues.

### FIELD OF THE INVENTION

The present invention relates to a method for the prevention of septic shock lethality using curcumin. The present invention also relates to a method for preventing septic shock lethality by reducing the severity of symptoms in an animal suffering from the said septic shock. More particularly, the method disclosed in the present invention inhibits neutrophil extravasation from the blood vessels into the underlying tissue responsible for inducing septic shock in a subject. At present, there is no suitable method to limit the lethality of septic shock syndrome. This is the first demonstration that a naturally occurring compound, curcumin, inhibits the lethality due to septic shock. Curcumin being a natural compound may have little side effects. The usage of curcumin may not only be restricted to septic shock, but also other conditions where infiltration of neutrophils plays a significant role.

### BACKGROUND OF THE INVENTION

Septic shock is a systemic response during which leukocytes infiltrate from blood vessels to underlying tissues and cause damage. Leukocytes keep circulating in the blood vessels, continually patrolling the body for foreign antigens. Lymphocytes recirculate from blood, through tissue, into lymph, and back to the blood. Granulocytes and monocytes cannot recirculate, but they emigrate from the blood vessels to the underlying tissue in response to molecular changes on the surface of blood vessels in case of an injury or an infection. Neutrophilic granulocytes are among the most abundant leukocytes in the bloodstream, and are among the first to appear at the sites of bacterial infection or injury. They are recruited locally to the sites of injury by various chemotactic agents including lipopolysaccharide derived from the bacterial cell walls, cytokines, and eicosanoids produced by local tissue monocytes and endothelial cells, and complement-derived anapylatoxins such as C3a and C5a. Once neutrophils have migrated into the tissue at the site of injury they release various mediators like peroxides and proteases for the clearance of the pathogen. This migration of leukocytes into the tissue is a part of the host response to protect an organ or tissue against damage (Reviewed by Springer T A, Cell, 76:301-304, 1994 and Paul L C et al., In Adhesion Molecules in Health and Disease, 1997).

In case of severe injury, infection or ischemia and reperfusion damage, there is a spill over of these activators into the systemic circulation that results in cellular activation. This causes indiscriminate neutrophil-endothelium adhesion and hence excessive infiltration of neutrophils into the tissue. These infiltrated neutrophils release mediators that besides clearing the pathogen also cause a damage to the host tissue.

One such condition arises upon severe infection with gram-negative bacteria. The lipopolysaccharide (LPS) that comprises the outer wall of the gram-negative bacteria activates various cells primarily macrophages, monocytes and other leukocytes. These activated cells release various mediators such as tumor necrosis factor (TNF-α), interleukin-1 (IL-1), IL-5, IL-8, also nitrous oxide, superoxide anions and lipid mediators (Michie H R et al., N. Engl. J. Med., 318:1481-1486, 1988). The release of these endogenous mediators leads to several pathophysiological reactions including fever, leukopenia, thrombocytopenia, intravascular coagulation and leukocyte infiltration in various organs that may ultimately lead to death. Thus, there is a systemic response to the invading pathogen and this is known as septic shock. Some of the visible symptoms of septic shock are shivering, fever, lethargy, diarrhea, watery eyes and ultimately leading to death (reviewed by Carlos J et al., Immunol. Today, 18: 329-334, 1997).

The lipopolysaccharide is cleared from the plasma by the liver. In rats, it is shown that kupfer cells are primarily responsible for the clearance of LPS from the blood, parenchymal and endothelial cells also contribute in the process. During septic shock, death is primarily caused due to liver damage which is caused by the excessive accumulation of neutrophils in the liver tissue (Jaeschke H et al., Am. J. Physiol. 261:G1051-G1056, 1991). Thus, inhibition of neutrophil infiltration and accumulation into the liver can prevent hepatocellular injury and prevent septic shock.

The mechanism of neutrophil induced liver injury consists of three steps: first sequestration of the neutrophils in the sinusoids, second transendothelial migration i.e. extravasation into the liver tissue and the third being adherence to the parenchymal cells. The extravasation of neutrophils into the tissue is necessary for the hepatocellular injury to occur. The sequestration of the neutrophils is not dependent on adhesion molecules whereas, transendothelial migration and adherence to parenchymal cells requires the adhesion molecules namely intercellular adhesion molecule-1 (ICAM-1), vascular cell adhesion molecule-1 (VCAM-1) and E-selectin (Essani N A et al., Hepatology, 21:1632-1639, 1995 and Oosten M V et al., Hepatology, 22:1538-1546, 1995). ICAM-1 plays an important role in the extravasation and adherence of leukocytes and its expression is highly upregulated during septic shock (Essani et al., Hepatology, 21:1632-1639, 1995). The involvement of ICAM-1 in septic shock has been demonstrated in ICAM-1 deficient mice where it has been shown that the mice are resistant to septic shock (Xu et al., J. Exp Med. 180:95, 1994).

Major advancements have been made in the development of antibiotics and medical intensive care technology in recent years, yet systemic response to infection remains a major health problem and a challenge in the new millenium (Dellinger R P et al., Infect. Dis. Clin. North. Am., 13:495-509, 1999). Priorities in the management of septic shock include rapid reversal of hypotension and hypoperfusion using compounds like dobutamine, dopamine etc, followed by empiric antibiotic therapy. Also selective removal of cytokines during continuous hemofiltration in septic patients with AN69 membranes is being carried out (Vriese A S et al., J. Am. Soc. Nephrol. 10:846-853, 1999). Variety of agents such as glucocorticoids, ibuprofen, antiendotoxin monoclonal antibodies, antagonists of platelet activating factor, of bradikynin or of interleukin receptor, and monoclonal anti-tumor necrosis factor (TNF) antibodies, inhibitors of complement factor 1 have undergone clinical trials for treatment of septic shock. All these major studies have yielded disappointing results (reviewed in Baumgartner J D et al., Drugs, 1999 57:127-132, 1999).

Curcumin (diferuloylmethane) is a major active component of turmeric (Curcuma longa). Curcumin has been reported to possess antioxidant property (Ammon et al., U.S. Pat. No. 5,401,777, 1995). It has been shown to possess anti-carcinogenic activity as it inhibits benzpyrene induced tumor initiation and phorbol ester induced tumor promotion (Huang M T et al., Cancer Res., 48:5941-5946, 1988). It also inhibits type 1 HIV-LTR directed gene expression and virus replication stimulated by TNF-α and phorbol ester (Li C J et al, Proc. Natl. Acad. Sci. USA 90:1839-1842, 1993). Recently, it has been reported to inhibit IL-5 and GM-CSF production by lymphocytes of bronchial asthmatics (Kobayashi T et al., Biochem. Pharmacol., 54: 819-824, 1997). Curcumin mediates some of its effects by inhibiting the binding of AP-1 to the DNA binding motif (Huang T S et al., Proc. Natl. Acad. Sci., 88:5292-5296, 1991) and it also prevents TNF-α dependent activation of NF-κB by preventing the degradation of IκB-α (Aggarwal, U.S. Pat. No. 5,891,924, 1999).

However, there is no prior art disclosure of any method whereby the efficacy of curcumin has been shown in animals i.e. in vivo for the prevention of systemic response, such as septic shock. This is the first demonstration that a natural compound can be used for alleviating the septic shock symptoms.

### OBJECTS OF THE INVENTION

The main object of the present invention is to provide a method of preventing lethality due to septic shock in an animal on administering the pharmacologically effective dose of curcumin.

Another object of the present invention is to provide a method of inhibiting the transmigration and infiltration i.e. extravasation of neutrophils into the tissue of the said animal in need of such a treatment by administering the pharmacologically effective dose of curcumin.

It is another object of the invention to provide a process for the inhibition of the lethality of septic shock by prevention of neutrophil extravasation from the blood vessels to the underlying tissue using a naturally occurring compound, curcumin with little side effects.

It is yet another object of the invention to provide a method for the prevention of neutrophil extravasation from blood vessels to underlying tissue using a naturally occurring compound, curcumin, that is inexpensive and readily available.

The usage of curcumin may not only be restricted to septic shock, but other conditions where infiltration of neutrophils plays a significant role.

### BRIEF DESCRIPTION OF THE DRAWINGS

The appended drawings illustrate preferred embodiments of the invention and thereof are not to be considered limiting in their scope.

In the drawing (s) accompanying this specification FIG. 1 shows the prevention of lethality by curcumin in mice injected with high dose of lipopolysaccharide. Mice were injected with 40 mg/kg lipopolysaccharide intraperitoneally on 0 day. In curcumin treated group mice were orally fed with 40 mg/kg curcumin 4 hrs and 2 hrs prior to, simultaneously and 4 hrs, 16 hrs, 24 hrs, 48 hrs and 72 hrs after injecting LPS. The mice were observed every two to three hours and the number of mice surviving noted in each group. Normal mice (-▪---) Curcumin treated & saline challenged (-Δ----), LPS injected (----▴---), Curcumin treated & LPS challenged (-♦----).

FIG. 2 shows representative of liver sections of mice from various treatment groups after LPS challenge. Mice were sacrificed 24 hrs after LPS challenge, their livers dissected out, fixed in bouins fixative, sectioned and stained with chloroacetylesterase to visualise neutrophils as detailed below. Representative liver sections from normal (A), LPS challenged (B) and curcumin treated & LPS challenged (C) mice. The arrows indicate clusters of neutrophils.

FIG. 3 shows inhibition of neutrophil adhesion to endothelial cells by curcumin. Endothelial cells grown to confluency in 96 well plates were incubated without or with indicated concentrations of curcumin for 1 hr followed by induction without (hatched bars) or with (closed bars) LPS (1 μg/ml) for 6 hrs. The cells were then incubated with human peripheral neutrophils for 1 hr. The amount of neutrophils adhering to the endothelial cell monolayers was measured by a colorimetric assay as described in Example 6.

### SUMMARY OF THE INVENTION

Upon systemic infection with gram-negative bacteria, the bacterial lipopolysaccharide activates various cells that release mediators, which activate the neutrophil-endothelium adhesion causing the neutrophils to infiltrate into the underlying tissue. An excessive infiltration of neutrophils causes damage to the host tissue.

The present invention demonstrates that curcumin is a potent inhibitor of neutrophil infiltration from the blood vessels into the underlying tissue. Treatment with curcumin prevents the infiltration of neutrophils into the liver tissue of the mice injected with gram-negative bacterial lipopolysaccharide. The neutrophils are arrested in the sinusoids and do not extravasate into the tissue.

Most importantly curcumin prevents the lethality in mice injected with gram-negative bacterial lipopolysaccharide. Also the mice treated with curcumin are less lethargic, do not suffer from diarrhea and their eyes are less watery; overall the severity of symptoms in the curcumin treated mice is much reduced compared to the mice injected with lipopolysaccharide alone.

The present invention also shows that curcumin blocks the adhesion of neutrophils to the vascular endothelial cells stimulated with lipopolysaccharide. These results also indicate that curcumin inhibits at a step of neutrophil-endothelium adhesion which is a prerequisite for the infiltration of the cells into the tissue. Thus the present invention shows that curcumin is able to prevent pathological conditions arising due to excessive infiltration of neutrophils into the tissues.

Accordingly, the present invention provides a method for the treatment of septic shock conditions in a subject by preventing lethality of said conditions and by reducing severity of symptoms, wherein said septic shock conditions are controlled by the prevention of neutrophil infiltration from blood vessels to underlying tissues, said method comprising administering orally a pharmacologically effective dose of curcumin to said subject at specified time intervals, wherein said effective dosage of curcumin ranges from 40 mg/kg to 60 mg/kg of body weight.

The process also relates to a method for the treatment of septic shock conditions in an animal by prevention of neutrophil infiltration from blood vessels to underlying tissues, said method comprising:

a) injecting intraperitoneally the bacterial lipopolysaccharide (LPS) solution to an animal, to induce septic shock,

b) administering orally a pharmacologically effective dose of curcumin prior to and after the said injection of LPS,

c) observing every two to three hours reduction in severity of septic shock symptoms selected from shivering, lethargy, fever, watery eyes, diarrhea and survival of an animal after 8 hours of administering LPS injection,

d) further probing the reduction in neutrophil infiltration from blood vessels to the underlying tissue by staining and microscopic examination for checking the extent of inflammation

In one embodiment of the invention, the pharmacologically effective dose of curcumin ranges from 40 mg/kg body weight to 60 mg/kg body weight.

In another embodiment to the present invention, the pharmacologically effective dose of curcumin is administered 2 to 4 hours prior to and simultaneous with LPS administration to an animal.

In yet another embodiment to the present invention, the pharmacologically effective dose of curcumin is administered at time intervals of 4, 16, 24, 48 and 72 hours after LPS administration.

In a further embodiment of the invention, the pharmacologically effective dose of curcumin is administered at time intervals of 3, 6, 9, 24 and 42 hours after LPS administration.

In yet another embodiment to the present invention the pharmacologically effective dose of curcumin may be administered orally as a suspension in pharmacologically acceptable non-toxic organic solvent or oil.

In still another embodiment the pharmacologically effective dose of curcumin is optionally administered orally alongwith an antioxidant preparation.

Other and further aspects, features, and advantages of the present invention will be apparent from the following description of the presently preferred embodiments of the invention given for the purpose of disclosure.

### DETAILED DESCRIPTION OF THE INVENTION

Septic shock is primarily caused upon severe infection with gram-negative bacteria. During septic shock death is mainly caused due to liver damage which is caused by the excessive accumulation of neutrophils in the liver tissue. Lipopolysaccharide that comprises the outer wall of the gram-negative bacteria activates various cell types including macrophages, monocytes and other leukocytes that release various mediators including tumor necrosis factor (TNF-α), interleukin-1 (IL-1), IL-6, IL-8, also nitrous oxide, superoxide anions and lipid mediators. These cytokines and mediators cause leukocyte infiltration in various organs and also upregulate the expression of cell adhesion molecules namely ICAM-1, VCAM-1 and E-selectin on the vascular endothelium. These adhesion molecules then help in the sequestration of the neutrophils from the blood vessels to the underlying tissue. The extravasation of neutrophils into the tissue is necessary for the hepatocellular injury to occur. Thus preventing the infiltration and accumulation of neutrophils into the liver and other tissues can prevent the host tissue damage and hence prevent lethality.

The present invention provides a method of reducing the severity of the symptoms in an animal suffering from systemic bacterial infection. For example, mice suffering from septic shock that are fed orally with curcumin are less lethargic, their eyes are less watery and they also suffer less from diarrhea.

The present invention also provides a method of preventing lethality due to septic shock induced by lipopolysaccharide in an animal in need of such a treatment by administering to the said animal a pharmacologically effective dose of curcumin orally. This dose has been found to be optimally 40 mg/kg to 60 mg/kg body weight, and it is required to be administered orally 4 hrs and 2 hrs prior to, simultaneously and 4 hrs, 16 hrs, 24 hrs, 48 hrs and 72 hrs after injecting LPS.

In the mice that had recovered from the septic shock upon treatment with curcumin, the administration of curcumin prior to LPS injection was not required, as the mice that were fed with curcumin alongwith, 3 hrs, 6 hrs, 9 hrs, 24 hrs and 42 hrs after LPS injection showed significant reduction in septic shock symptoms, also recovered early and all the animals survived.

This indicates that prior administration of curcumin preconditioned the mice and hence was beneficial.

The present invention is also, directed to a method of inhibiting the transmigration and infiltration of neutrophils from the blood vessels into the tissue of an animal in need of such a treatment by administering to the said animal a pharmacologically effective dose of curcumin. This dose has been found to be optimally 40 mg/kg to 60 mg/kg and it needs to be administered 4 hrs and 2 hrs prior to, simultaneously and 4 hrs, 16 hrs, 24 hrs, 48 hrs and 72 hrs after injecting LPS.

Curcumin acts to prevent septic shock by preventing the adherence of neutrophils to the endothelial cells. It thus prevents their transmigration from the blood vessels to the underlying tissue. This in turn prevents the accumulation of the neutrophils in the liver and hence prevents the hepatocellular injury caused by the neutrophils.

The following examples are given for the purpose of illustrating various embodiments of the invention and are rot meant to limit the present invention in any fashion.

### EXAMPLE 1

### Materials

TNF-α, anti-ICAM-1 (BBA3), anti-VCAM-1 (BBA6) and anti-E-selectin (BBA1) antibodies were purchased from R & D Systems, California. M199, 1-glutamine, penicillin, streptomycin, amphotericin, endothelial cell growth factor, trypsin, Pucks saline, HEPES, ficoll-hypaque, tetra methyl benzidine, cetitrimethyl ammonium bromide, Lipolysaccharide from E. coli serotype 0127:B7, napthol-ASD chloroacetate esterase staining kit and 3-amino-1,2,4 triazole were purchased from Sigma Chemical Co., USA.. Foetal calf serum was purchased from Biological industries, Israel.

### EXAMPLE 2

### Procedure for Animal Experiments

In septic shock experiments, age-and body weight-matched (25-30 g) female swiss albino mice were injected intraperitoneally with 40 mg/kg LPS solubilised in 200 μl sterile 0.9% saline. Control animals received equal volumes of saline. For groups that were treated with curcumin, the mice were fed orally with 20 mg/kg to 60 mg/kg body weight curcumin suspended in olive oil (20 mg/ml) at various times prior to and after injecting LPS. The mice were fed with curcumin 4 hrs and 2 hrs prior to, simultaneously and 4 hrs, 16 hrs, 24 hrs, 48 hrs and 72 hrs after injecting LPS. Following this, the mice were observed every 2-3 hours after LPS injection with respect to septic shock symptoms such as fever, lethargy, diarrhea, watery eyes and survival.

### EXAMPLE 3

### Histology

For histological examinations the mice were sacrificed after 24 hrs of LPS administration, their liver were dissected out and small portions were fixed in bouin's fixative (formalin-picric acid-acetic acid). The fixed portions of the liver were then embedded in paraffin. Paraffin-embedded sections of the liver were cut 5 μM and placed on Mayer's albumin-coated slides. The tissue sections were dewaxed with xylene, and rehydrated through graded concentrations of ethanol. Polymorphonuclear cells were stained using napthol AS-D chloroacetae esterase staining technique according to manufacturer's protocol, Sigma Chemical Co., USA.

### EXAMPLE 4

### Cells and Cell Culture

Primary endothelial cells were isolated from human umbilical cord. For isolation of the cells the umbilical cord vein was canulated and then perfused with phosphate buffer saline to remove blood and then incubated with 0.125% trypsin for 15 mins at 37° C. The vein was then perfused with PBS and the cells were collected. The endothelial cells thus obtained were cultured in M 199 medium supplemented with 20% heat inactivated fetal calf serum, 2 mM 1-glutamine, 100 units/ml penicillin, 100 μg/ml streptomycin, 0.25 μg/ml amphotericin and endothelial cell growth factor (50 μg/ml) supplemented with heparin (5 U/ml) in gelatin coated T-75 cm² flasks. The cells were subcultured by dislodging with 0.125% trypsin-0.01 M EDTA solution in Pucks saline and HEPES buffer. The viability of endothelial cells in culture was checked by trypan blue exclusion test and the purity was determined by E-selectin staining. The cells were used within first four passages.

### EXAMPLE 5

### Neutrophil Isolation

Neutrophils were isolated from peripheral blood of healthy individuals as previously described (Clark R A, In Curr. Prot. in Immunol., p7.23.1). Briefly, 10 ml of the peripheral blood was collected in heparin solution (20 U/ml final concentration) and erythrocytes were removed by sedimentation with 6% dextran solution. The white blood cell rich plasma layer was collected and layered over ficoll-hypaque solution followed by centrifugation for 20 minutes at 300×g at 20° C. The top saline layer and the ficoll-hypaque layer were aspirated leaving the neutrophil/RBC pellet. The residual RBC's were removed by hypotonic lysis. The isolated cells were washed with PBS and resuspended in PBS containing 5 mM glucose, 1 mM CaCl₂ and 1 mM MgCl₂ at a final concentration of 6×10⁵ cells/ml. This procedure usually resulted in approximately 95% neutrophils and the cell viability was more than 95% as detected by trypan blue exclusion test.

### EXAMPLE 6

### Cell Adherence Assay

Adhesion of neutrophils to endothelial monolayers was assayed as described previously (Dobrina A et al., J. Clin. Invest., 88:20-26 1991). Briefly, the endothelial cells were plated in 96 well, flat bottom, gelatin coated culture plates at a density of 2×10⁴ cells/well and allowed to adhere for 24 hrs in a humidified chamber maintained at 37° C. and 5.0% CO₂. The cells were incubated without or with 10 μM to 40 μM curcumin for 1 hr followed by induction with LPS (1 μg/ml) for 6 hrs and washed with PBS twice. The isolated neutrophils (6×10⁴/well) were added to the endothelial monolayers and incubated for 1 hr at 37° C. Non-adherent neutrophils were removed by washing the wells with PBS thrice. Adherent neutrophils were assayed calorimetrically by adding a substrate solution (75 μl/well) consisting of 2 mM tetramethylbenzidine in 0.1 M sodium acetate buffer (pH 4.2) containing 0.1% cetitrimethyl ammonium bromide as peroxidase solubilising agent. Adding a selective eosinophil peroxidase inhibitor, 3-amino-1,2,4 triazole (1 mM) to the substrate solution, abolished the interference by few contaminating eosinophils. After 2 min of incubation with substrate solution 0.7 mM hydrogen peroxide (75 μl/well) was added. The reaction was stopped by adding 2 N H₂SO₄ (50 μl/well). The absorbance was determined at 450 ηM using an automated microplate reader (Anthos Labtech HT2, Austria).

### EXAMPLE 7

### Protection of Mice from Septic Shock by Curcumin

Intraperitoneal injection of high doses of LPS (40 mg/kg) induced lethal endotoxin shock in mice. The mice demonstrated a series of symptoms including shivering, lethargy, fever, watery eyes due to enhanced vasopermeability, diahhorea and ultimately death. The death occurred within 24-48 hrs after receiving LPS. To test whether curcumin protects mice injected with LPS from septic shock, we treated mice with varying doses of curcumin 4 hrs and 2 hrs prior to, simultaneously and 4 hrs, 16 hrs, 24 hrs, 48hrs and 72 hrs after injecting LPS. Low dose of curcumin was not able to protect the mice, the optimal dose of curcumin was found to be 40 mg/kg to 60 mg/kg (as shown in Table 1). Interestingly, the mice that were treated with curcumin were less lethargic, their eyes were less watery (as shown in the photographs) and suffered less from diarrhea. Overall, severity of the septic shock symptoms in treated group was much less in comparison with control groups (as shown in Table 2). The mice start recovering within 48 to 72 hrs. Most importantly, 70% of the mice treated with 40 mg/kg to 60 mg/kg curcumin survived from death (FIG. 1). When the recovered mice were subjected to LPS injection with only simultaneous and post administration of curcumin they showed significant reduction in severity of septic shock symptoms, recovered early and all survived death.

### EXAMPLE 8

### Prevention of Liver Damage and Inhibition of Infiltration of Leukocytes in Liver by Curcumin

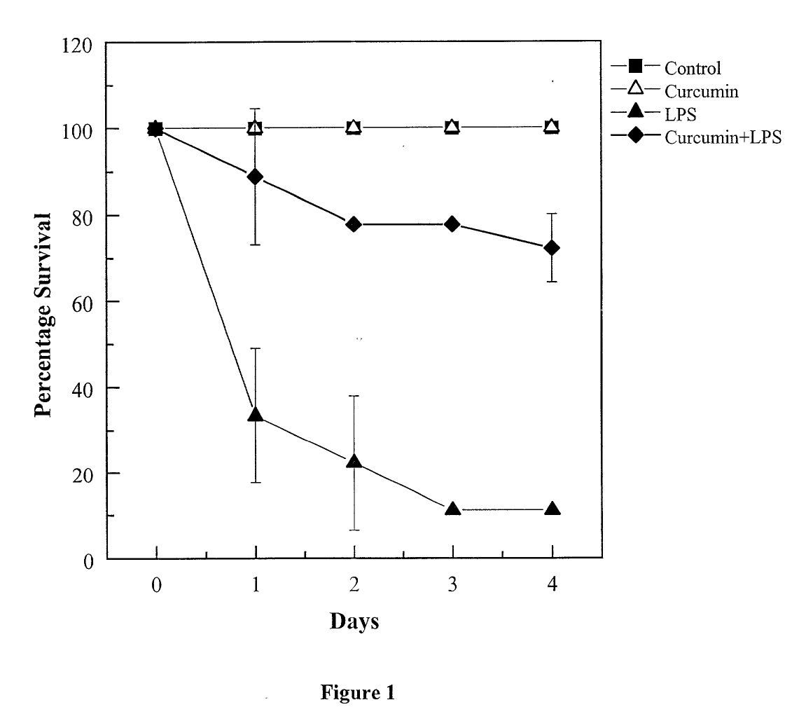

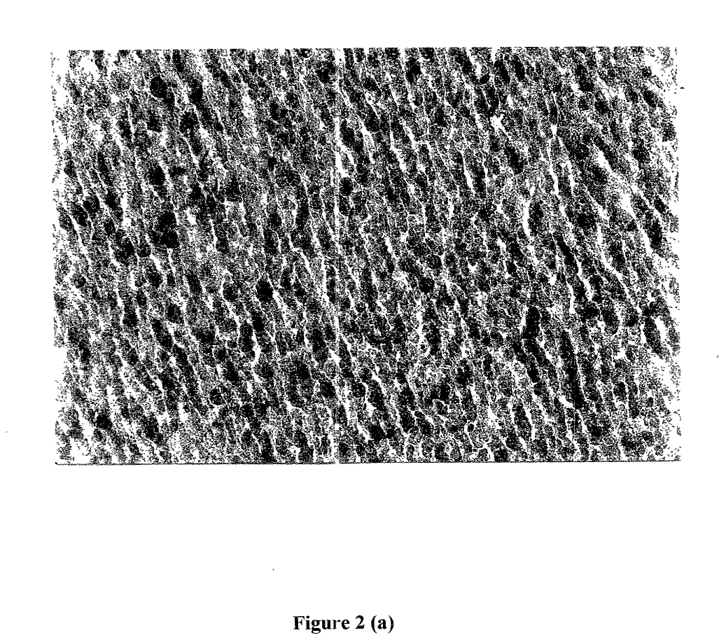

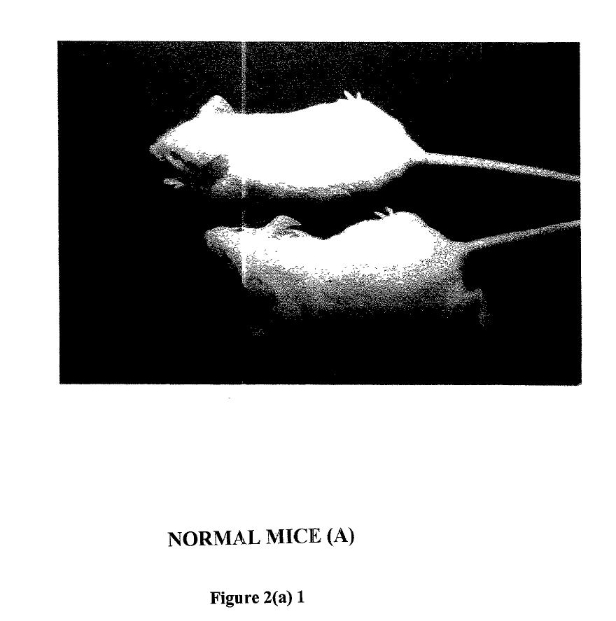

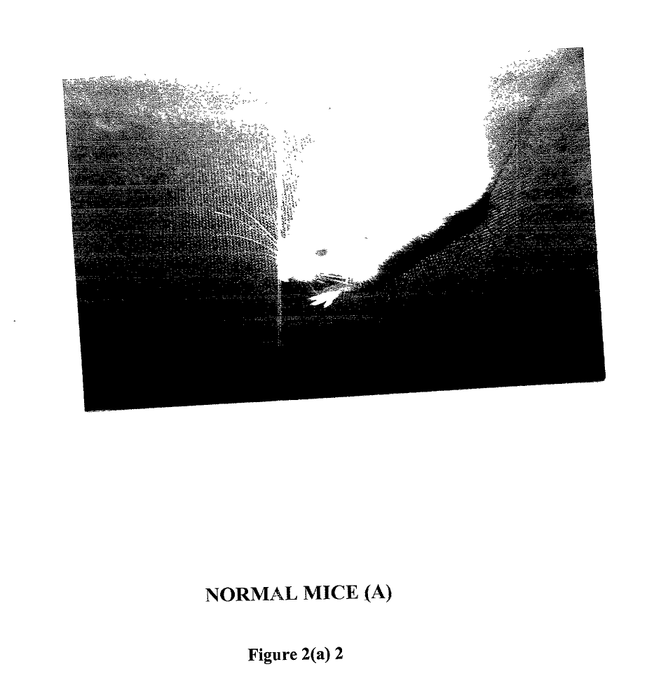

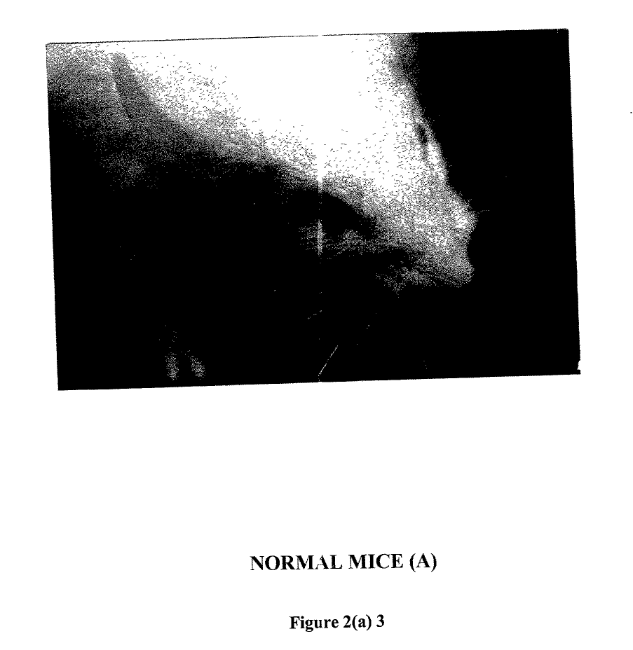

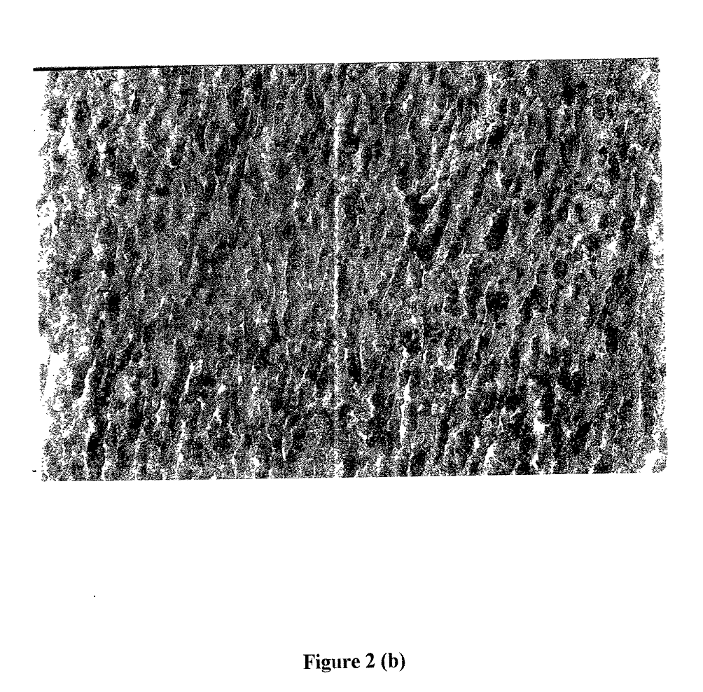

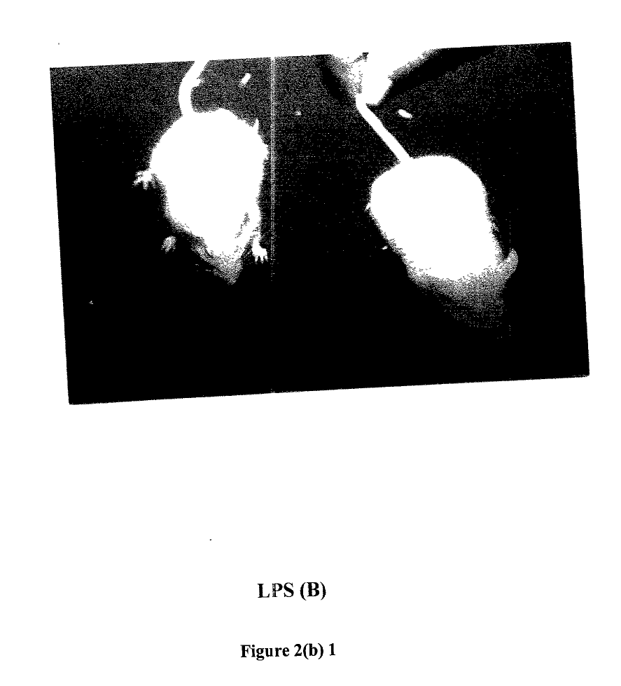

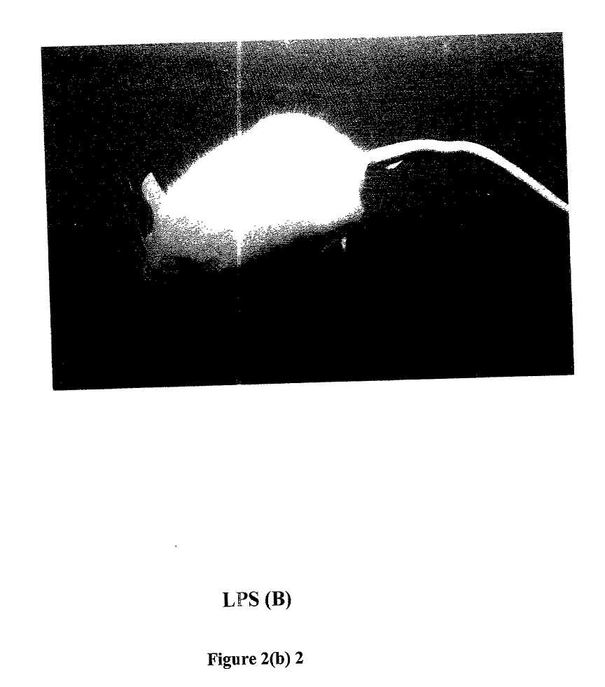

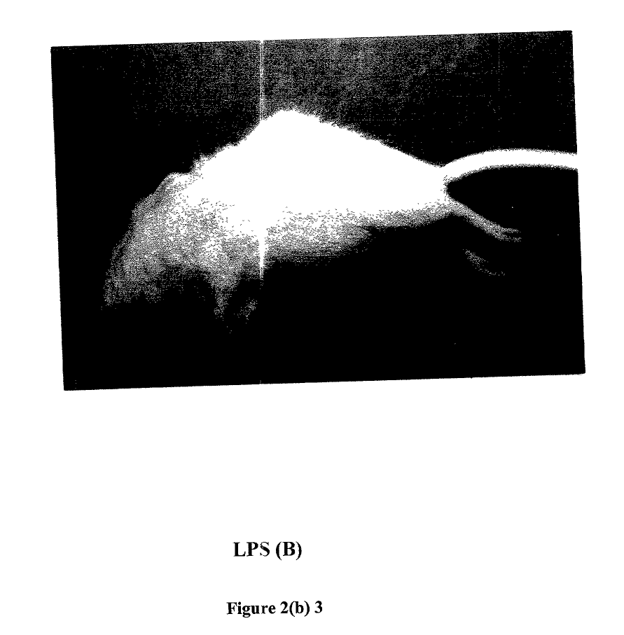

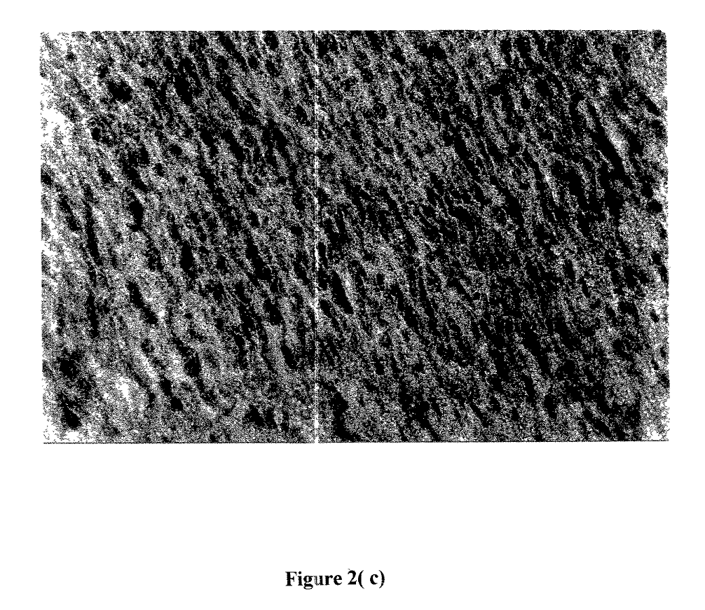

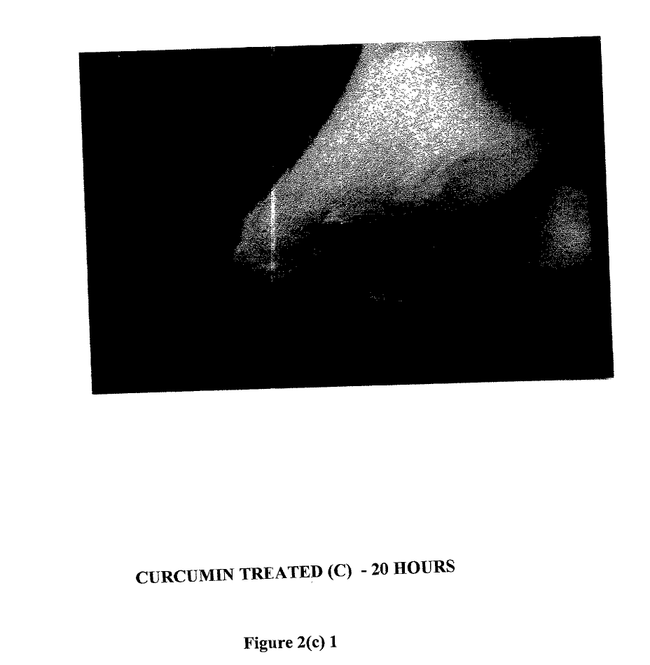

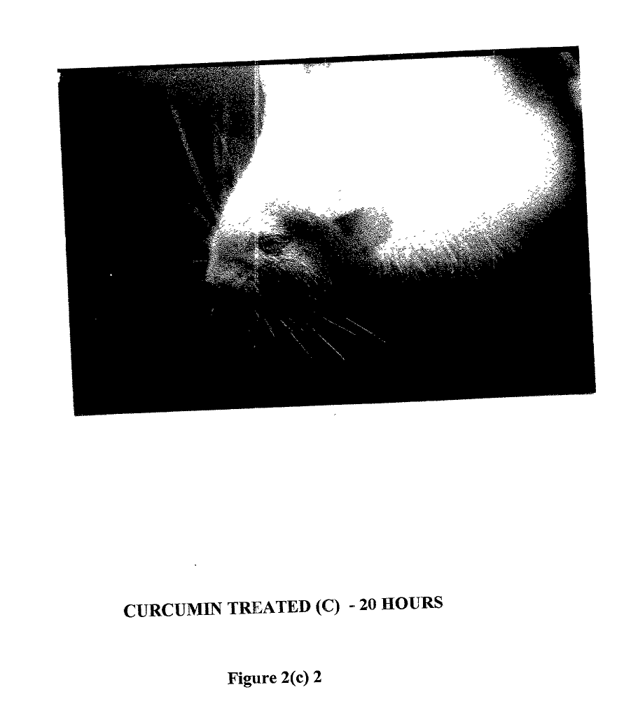

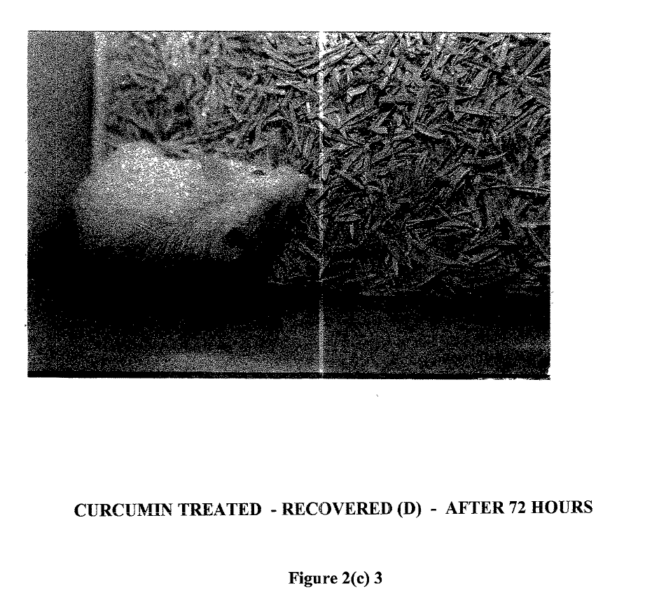

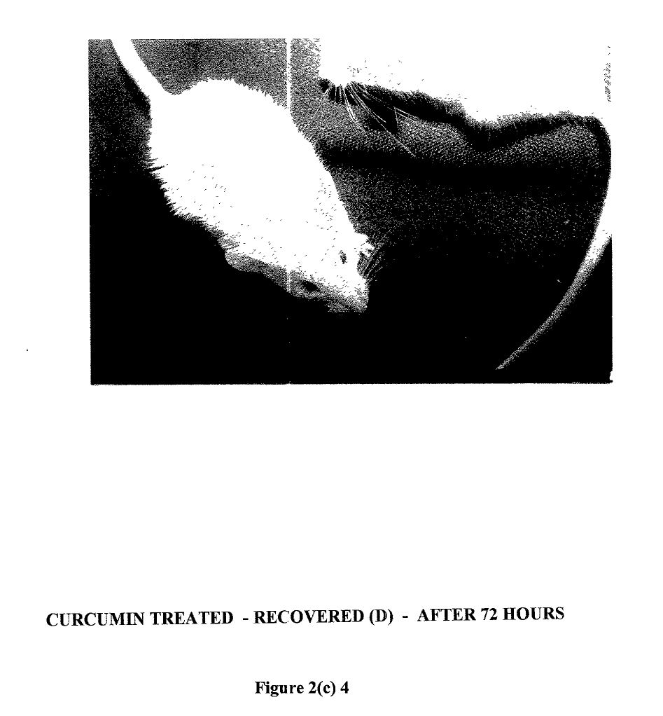

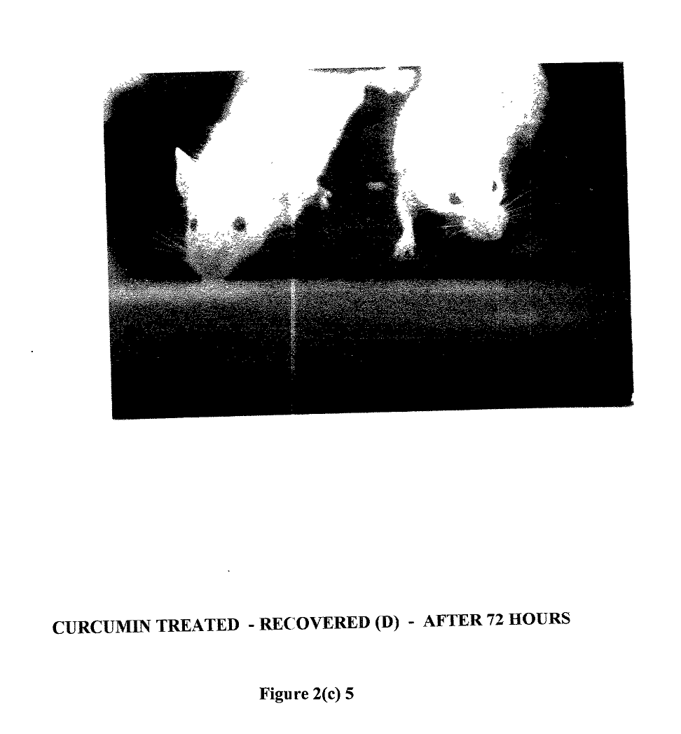

Death in septic shock is primarily caused by liver damage due to the excessive infiltration of leukocytes (5). To find the mechanism by which curcumin protects mice from septic shock, we examined the accumulation of leukocytes into the liver of normal, LPS injected and curcumin treated mice histologically by chloroacetate esterase staining as described in Example 3. An excessive accumulation of neutrophils was observed in the liver of LPS treated mice. Most of the neutrophils extravasated from blood vessels into the surrounding tissue and formed clusters of three to five, often larger aggregates were also found (FIG. 2b). In contrast, the extravasation of neutrophils in the liver of curcumin treated mice was found to be much reduced compared to the untreated mice (FIG. 2, compare b and c). The neutrophils accumulated in the hepatic venules and their transendothelial migration was prevented. The liver of the curcumin treated mice had fewer clusters of neutrophils that were mostly scattered. Interestingly, the neutrophils were mostly present in the hepatic venules but did not infiltrate into the tissue (FIG. 2c). It has also been shown that extravasation of neutrophils and their adherence to the parenchyma cells is essential for the liver damage. As curcumin prevents the transendothelial migration of neutrophils thus there is less damage to the liver of curcumin treated mice.

### EXAMPLE 9

### Inhibition of Adhesion of Neutrophils to Endothelial Cells by Curcumin

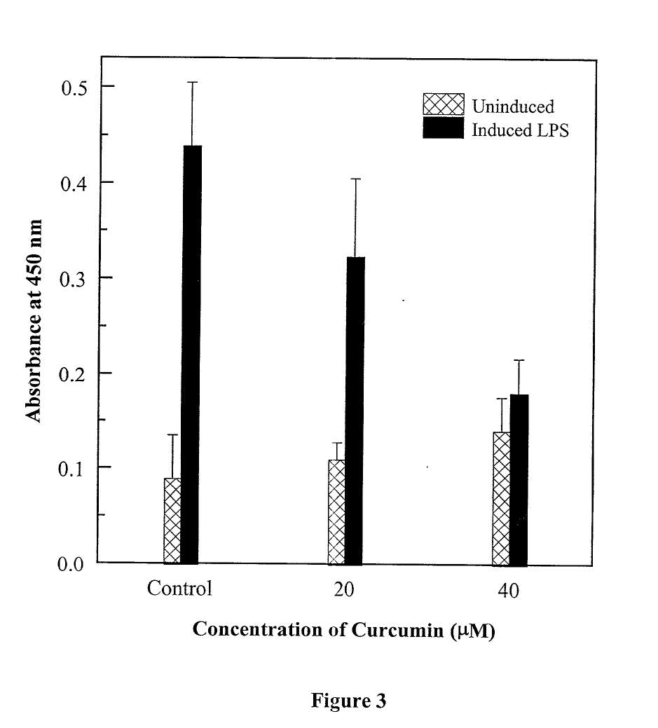

As curcumin prevented transendothelial migration of neutrophils in liver of treated mice thus to confirm the inhibition of neutrophil adhesion to endothelial cells by curcumin, we tested the adhesion of peripheral blood neutrophils to the endothelial cell monolayers in presence of 10 μM to 40 μM curcumin. As shown in FIG. 3 the adhesion of neutrophils to the unstimulated endothelial cells was found to be low and there was a three to four fold upregulation of neutrophil adhesion to endothelial cells on stimulation with LPS. Although curcumin did not affect the adhesion of neutrophils to unstimulated endothelial monolayers, it blocked the neutrophil adhesion to the LPS stimulated endothelial cells in a concentration dependent manner with almost complete inhibition at a concentration of 40 μM (FIG. 3).

### The Main Advantages of the Present Invention are

1. This is the first demonstration where a naturally occurring compound, curcumin, has been shown to inhibit the lethality due to septic shock by prevention of neutrophil extravasation from the blood vessels to the underlying tissue.

2. Curcumin being a natural compound has little side effects.

3. Curcumin is inexpensive and readily available.

4. The usage of curcumin may not only be restricted to septic shock, but other conditions where infiltration of neutrophils plays a significant role.

References:

Aggarwal, B. B. 1999. Curcumin inhibition of NF-κB activation. U.S. Pat. No. 5,891,924.

Ammon, H. P. T., Hasan, S. and Sameul, O. 1995. Use and preparations of curcuma plants. U.S. Pat. No. 5,401,777.

Baumgartner, J. D. and Calandra T. 1999. Treatment of sepsis: past and future avenues. Drugs 1999 57:127.

Carlos, J., Ramos, G. and Bluethmann, H 1997. Molecules and mechanisms operating in septic shock: lessons from knockout mice. Immunol. Today. 18: 329.

Clark, R. A., and Nauseef, W. M. 1996. Preparation and functional analysis of other human lymphoid and non lymphoid cells. In Curr. Prot. in Immunol. Vol. 2. Coligan, J. E., Kruisbeek, A. M., Margulies, D. H., Shevach E. M. and Strober, W. eds. John Wiely & Sons, Inc. USA. p 7.23.1

Dellinger R P. 1999. Current therapy for sepsis. Infect Dis Clin North Am 13:495

Dobrina, A., Menegazzi, R., Carlos, T. M., Nardon, E., Cramer, R., Zacchi, T., Harlan, J. M. and Patriarca, P. 1991. Mechanisms of the eosinophil adherence to cultured vascular endothelial cells. J. Clin. Invest. 88:20.

Essani, N. A., Fischer, M. A., Farhood, A, Manning, A. M., Smith, C. W. and Jaeschke H. 1995. Cytokine-induced upregulation of hepatic intercellular adhesion molecule-1 messenger RNA expression and its role in the pathophysiology of murine endotoxin shock and acute liver failure. Hepatology. 21:1632.

Haung, M. T., Smart, R, C., Wong, C. Q. and Conney, A. H. 1988. Inhibitory effect of curcumin, chlorogenic acid, caffeic acid, and ferulic acid on tumor promotion in mouse skin by 12-0-tetradecanoylphorbol-13-acetate. Cancer Res. 48:5941.

Huang, T. S., Lee, S. C. and Lin. J. K. 1991. Suppression of c-Jun/AP1 activation by an inhibitor of tumor promotion in mouse fibroblast cells. Proc. Natl. Acad. Sci. USA. 88:5292.

In Adhesion Molecules in Health and Disease. 1997. Paul, L. C. and Issekutz, T. B. eds. Marcel Dekker, Inc., New York.

Jaeschke, H., Farhood, A. and Smith, C. W. 1991. Neutrophil induced liver injury in endotoxin shock is a CD11b/CD18 dependent mechanism. Am. J. Physiol. 261:G1051.

Kobayashi, T., Hashimoto, S. and Horie, T. 1997. Curcumin inhibition of dermatophagoides farinea-induced interleukin-5 (IL-5) and granulocyte macrophage-colony stimulation factor (GM-CSF) production by lymphocytes from bronchial asthmatics. Biochem. Pharmacol. 54: 819

Li, C. J., Zhang, L. J., Dezube, B. J., Crumpacker, C. S. and Pardee, A. B. 1993. Three inhibitors of type 1 human immunodeficiency virus long terminal repeat-directed gene expression and virus replication. Proc. Natl. Acad. Sci. USA 90:1839.

Michie, H. R., Maogue, K. R., Spriggss. D. R. 1988. Detection of circulating tumor necrosis factor after endotoxin administration. N. Engl. J. Med. 318:1481.

Oosten, M. V., Bilt, E. V., Vries, H. E., Berkel, T. J. C. and Kuiper, J. 1995. Vascular adhesion molecule-1 and intercellular adhesion molecule-1 expression on the rat liver cells after lipopolysaccharide administration in vivo. Hepatology. 22: 1538.

Springer, T. A. 1994. Traffic signals for lymphocyte recirculation and leukocyte emigration: the multistep paradigm. Cell 76: 301.

Vriese A. S., Colardyn F. A., Phillippe J. J., Vanholer R. C., Sutter J. H. and Lameire N. H. 1999. Cytokine removal during continuous hemofiltration in septic patients. J. Am. Soc. Nephrol. 10:846

Xu, H., Gonzalo, J. A., Pierre, Y. S., Williams, I. R., Kupper, T. S., Cotran, R. S., Springer, T. A., Carlos, J. and Ramos, G. 1994. Leukocytosis and resistance to septic shock in intercellular adhesion molecule 1-deficient mice. J. Exp. Med. 180:95.

TABLE 1
Dose dependent effect of curcumin on survival of the mice:
The mice were treated with varying dose of curcumin prior
to and after injecting LPS (40 mg/kg). Following this the
survival of mice was recorded.

| Dose of Curcumin (mg/kg | |
|---|---|
| body weight) | Percentage Survival |
| 20 | 0 |
| 40 | 70 |
| 60 | 70 |

We claim:

1. A method for the treatment of septic shock conditions in a subject by preventing lethality of said conditions and by reducing severity of symptoms, wherein said septic shock conditions are controlled by the prevention of neutrophil infiltration from blood vessels to underlying tissues, said method comprising administering orally a pharmacologically effective dose of curcumin to said subject at specified time intervals, wherein said effective dosage of curcumin ranges from 40 mg/kg to 60 mg/kg of body weight.

2. A method for the treatment of septic shock conditions in an animal wherein the said method comprises:
a) injecting intraperitoneally the bacterial lipopolysaccharide (LPS) solution to an animal, preferably mice of sound health, to induce septic shock,
b) administering orally a pharmacologically effective dose of curcumin prior to and after the said injection of LPS,
c) observing every two to three hours reduction in severity of septic shock symptoms selected from shivering, lethargy, fever, watery eyes, diarrhea and survival of an animal after 8 hours of administering LPS injection,
d) further probing the reduction in neutrophil infiltration from blood vessels to the underlying tissue by staining and microscopic examination for checking the extent of inflammation.

3. A method claimed in claim 2, wherein the pharmacologically effective dose of curcumin ranges from 40 mg/kg to 60 mg/kg body weight.

4. A method as claimed in claim 2, wherein the pharmacologically effective dose of curcumin is administered two to four hours prior to and simultaneous with LPS administration.

5. A method as claimed in claim 2, wherein the pharmacologically effective dose of curcumin is administered at time intervals of 4, 16, 24, 48 and 72 hours after LPS administration.

6. A method as claimed in claim 2, wherein the pharmacologically effective dose of curcumin is administered at time intervals of 3, 6, 9, 24 and 42 hours after LPS administration

7. The method claimed in claim 2, wherein the said curcumin is administered orally as a suspension in pharmacologically acceptable non-toxic organic solvent or oil.

8. A process as claimed in claim 2 wherein the pharmacologically effective dose of curcumin is optionally administered orally along with an antioxidant preparation.
